(19) United States
(12) Patent Application Publication
(10) Pub. No.: US 2025/0236909 A1
(43) Pub. Date: Jul. 24, 2025

(54) METHODS FOR DETECTING ALLELE DOSAGES IN POLYPLOID ORGANISMS

(71) Applicant: Life Technologies Corporation

(72) Inventors: Krishna Reddy Gujjula, Georgetown, TX (US); Haktan Suren, Leander, TX (US); Cheng-Zong Bai, Fremont, CA (US)

(21) Appl. No.: 19/074,700

(22) Filed: Mar. 10, 2025

Related U.S. Application Data
(63) Continuation of application No. PCT/US2023/073826, filed on Sep. 11, 2023.
(60) Provisional application No. 63/375,267, filed on Sep. 12, 2022.

(51) Int. Cl. C12Q 1/6858

(52) U.S. Cl. CPC C12Q 1/6858

(57) ABSTRACT

A method for determining a genotype of a sample of a polyploid organism, may include: amplifying nucleic acid sequences at targeted locations in a sample genome by a panel targeting a plurality of SNP markers to generate sequence reads; mapping the sequence reads to a reference genome for the polyploid organism; detecting variants in the aligned sequence reads to produce detected variants, wherein the detected variants include detected SNP's corresponding to the SNP markers; determining a probability for each alternate allele dosage of a plurality of possible allele dosages for a corresponding detected SNP, wherein the number of possible allele dosages is equal to a ploidy of the SNP marker plus one; and selecting the alternate allele dosage with a maximum probability value to provide an estimated allele dosage corresponding to the SNP marker, wherein the estimated allele dosage is indicative of the genotype for the SNP marker.

### CROSS-REFERENCE TO RELATED APPLICATIONS

This application is a continuation of International Patent Application no. PCT/US2023/073826, filed Sep. 11, 2023, which claims the benefit under 35 U.S.C. § 119(e) of U.S. Provisional Application No. 63/375,267, filed Sep. 12, 2022. The entire contents of the aforementioned applications are incorporated by reference herein.

### FIELD

The present application relates to methods, systems, and computer-readable media for detecting allele dosages for single nucleotide polymorphisms (SNP) markers in samples of polyploid organisms using targeted sequencing and next-generation sequencing (NGS) technology.

### BRIEF DESCRIPTION OF THE DRAWINGS

The novel features of the invention are set forth with particularity in the appended claims. A better understanding of the features and advantages will be obtained by reference to the following detailed description that sets forth illustrative embodiments and the accompanying drawings of which:

FIG. 1 is a block diagram of an example process for determining genotypes of polyploid SNP markers.

FIG. 2 is a block diagram of an example process for determining the allele dosage.

FIG. 3 is a schematic diagram of an exemplary system for reconstructing a nucleic acid sequence, in accordance with various embodiments.

FIG. 4 is an example of a block diagram of an analysis pipeline for signal data obtained from a nucleic acid sequencing instrument.

### DETAILED DESCRIPTION

In accordance with the teachings and principles embodied in this application, new methods, systems and non-transitory machine-readable storage medium are provided for determining allele dosages for SNP markers in samples of polyploid organisms. The teachings further provide for determining allele dosages on a per sample per marker basis.

As used herein, allele dosages correspond to alternate alleles. An alternate allele dosage represents the number of copies which contain the alternate allele. For example, in a tetraploid organism, generally there are four copies of a chromosome. Hence an alternate allele for a marker can five states, 0, 1, 2, 3 or 4. For a given marker in a sample, if the alternate allele dosage is 0 then none of the copies contain the alternate allele, if alternate allele dosage is 1 then one copy out of four chromosomes contains the alternate allele and so forth.

Phenotype is influenced by both environmental factors and genotypic states of the trait loci. In polyploid organism, genotypic states is often inferred by allele dosages. Such allele dosages are used in Genome Wide Association Studies (GWAS) to discover and rank genomic loci related to traits of interest and these loci are later on used to select the individuals for inclusion in agricultural breeding program. This process is known as marker assisted selection (MAS). MAS improves the breeding efficiency by substantially reducing the breeding cycle and cost.

An advantage of the method described herein includes providing allele dosages for markers in an individual sample, without considering information from other samples in the multiplexed run. Hence the method is not susceptible to bias in allele dosage estimation when there is a low number of samples or if a particular marker is monomorphic in all the multiplexed samples. A further advantage of the method is allowing for marker level parameter customization, which enables greater accuracy in the estimate of allele dosage.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA.

In various embodiments, a “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The term “allele” as used herein refers to a genetic variation associated with a gene or a segment of DNA, i.e., one of two or more alternate forms of a DNA sequence occupying the same locus.

The term “locus” as used herein refers to a specific position on a chromosome or a nucleic acid molecule. Alleles of a locus are located at identical sites on homologous chromosomes.

The phrase “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to: single nucleotide polymorphisms (SNPs), copy number variations (CNVs), insertions/deletions (Indels), inversions, etc.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

As defined herein, “sample” and its derivatives, is used in its broadest sense and includes any specimen, culture and the like that is suspected of including a target. In some embodiments, the sample comprises DNA, RNA, PNA, LNA, chimeric, hybrid, or multiplex-forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen.

The phrase “sample genome” can denote a whole or partial genome of an organism.

As used herein, the terms “adapter” or “adapter and its complements” and their derivatives, refers to any linear oligonucleotide which can be ligated to a nucleic acid molecule of the disclosure. Optionally, the adapter includes a nucleic acid sequence that is not substantially complementary to the 3′ end or the 5′ end of at least one target sequences within the sample. In some embodiments, the adapter is substantially non-complementary to the 3′ end or the 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. An adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

As used herein, “DNA barcode” or “DNA tagging sequence” and its derivatives, refers to a unique short (e.g., 6-14 nucleotide) nucleic acid sequence within an adapter that can act as a ‘key’ to distinguish or separate a plurality of amplified target sequences in a sample. For the purposes of this disclosure, a DNA barcode or DNA tagging sequence can be incorporated into the nucleotide sequence of an adapter.

In some embodiments, the disclosure provides for amplification of multiple target-specific sequences from a population of target nucleic acid molecules. In some embodiments, the method comprises hybridizing one or more target-specific primer pairs to the target sequence, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target sequences. In some embodiments, the digesting includes partial digesting of one or more of the target-specific primers from the amplified target sequence. In some embodiments, the amplified target sequences can be ligated to one or more adapters. In some embodiments, adapters can include one or more DNA barcodes or tagging sequences. In some embodiments, amplified target sequences once ligated to an adapter can undergo a nick translation reaction and/or further amplification to generate a library of adapter-ligated amplified target sequences.

In some embodiments, the methods of the disclosure include selectively amplifying target sequences in a sample containing a plurality of nucleic acid molecules and ligating the amplified target sequences to at least one adapter and/or barcode. Adapters and barcodes for use in molecular biology library preparation techniques are well known to those of skill in the art. The definitions of adapters and barcodes as used herein are consistent with the terms used in the art. For example, the use of barcodes allows for the detection and analysis of multiple samples, sources, tissues or populations of nucleic acid molecules per multiplex reaction. A barcoded and amplified target sequence contains a unique nucleic acid sequence, typically a short 6-15 nucleotide sequence, that identifies and distinguishes one amplified nucleic acid molecule from another amplified nucleic acid molecule, even when both nucleic acid molecules minus the barcode contain the same nucleic acid sequence. The use of adapters allows for the amplification of each amplified nucleic acid molecule in a uniformed manner and helps reduce strand bias. Adapters can include universal adapters or propriety adapters both of which can be used downstream to perform one or more distinct functions. For example, amplified target sequences prepared by the methods disclosed herein can be ligated to an adapter that may be used downstream as a platform for clonal amplification. The adapter can function as a template strand for subsequent amplification using a second set of primers and therefore allows universal amplification of the adapter-ligated amplified target sequence. In some embodiments, selective amplification of target nucleic acids to generate a pool of amplicons can further comprise ligating one or more barcodes and/or adapters to an amplified target sequence. The ability to incorporate barcodes enhances sample throughput and allows for analysis of multiple samples or sources of material concurrently.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence.

The phrase “next generation sequencing,” or NGS, refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

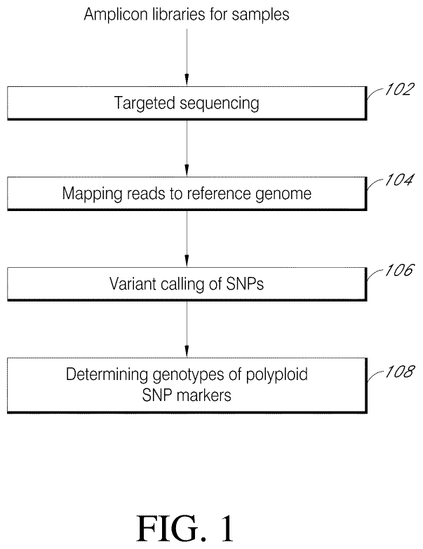

FIG. 1 is a block diagram of an example process for determining genotypes of polyploid SNP markers. Selectively amplifying nucleic acid sequences at targeted locations in the sample genome by a panel targeting SNP marker regions may produce amplicon libraries for one or more test samples. For example, the Applied Biosystems AgriSeq HTS Library Kit (Thermo Fisher Scientific catalog nos. A34144 and A34143) provides high-throughput preparation of amplicon libraries for targeted genotyping-by-sequencing (GBS) applications in agrigenomics. The amplicon libraries of multiple samples may be barcoded to distinguish different samples that are sequenced simultaneously in a single sequencing run. In step 102, the amplicons of the sample libraries are sequenced by a nucleic acid sequencing device, such as a next generation sequencing device, to produce a plurality of sequence reads. A plurality of samples from one or more polyploid organisms may be sequenced simultaneously in a single run to produce a plurality of sequence reads. In step 104, the sequence reads are mapped to a reference genome for the organism to produce the aligned sequence reads. In the variant calling step 106, a processor receives aligned sequence reads resulting from the targeted sequencing. The aligned sequence reads can be retrieved from a file using a BAM file format, for example. The aligned sequence reads may correspond to a plurality of targeted SNP marker regions. The variant calling step 106 may be configured by one or more variant caller parameters. The variant calling step 106 may provide an observed population of variants, such as SNPs, detected in the aligned sequence reads. In some embodiments, the variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which incorporated by reference herein in its entirety. Other variant detection methods may be used. In various embodiments, a variant caller can be configured to communicate variants called for a sample genome as a *.vcf, *.gff, or *.hdf data file. The called variant information can be communicated using any file format as long as the called variant information can be parsed and/or extracted for analysis.

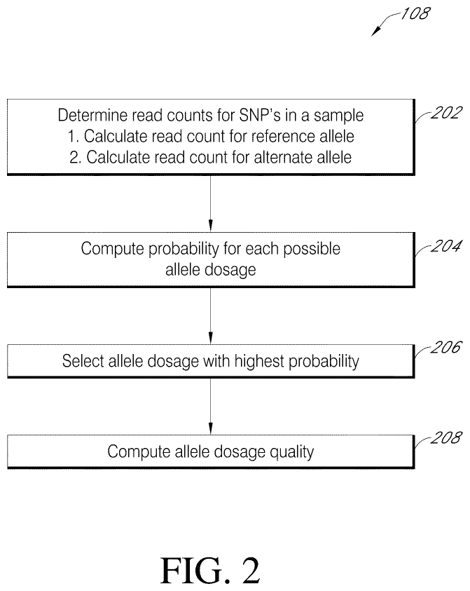

FIG. 2 is a block diagram of an example process for determining the allele dosage per SNP marker per sample. The allele dosage per SNP per sample may be determined for a plurality of samples from one or more polyploid organisms. Step 202 may determine the read counts supporting the reference allele and alternate allele of observed populations of SNPs for each marker in each sample. The user may define the reference allele and alternate allele. For example, the variant caller may generate the read count for the reference allele and the read count for the alternate allele for each SNP marker in each sample. Step 204 may compute a probability for each possible allele dosage for each SNP marker in each sample. For example, for a tetraploid organism, a SNP can have a total of 5 possible allele dosages corresponding to 0, 1, 2, 3, and 4. For example, a probability model fit to read count data observed at a position in a given sequence read may be generated for hypothesized alleles. An a posteriori probability distribution f_{APP} is given by the expression:

$${f}_{A P P} ( {p}_{H} = f ) = Beta ( k + 1 , n - k + 1 ) \qquad ( 1 )$$

where (p_{H}=f) is the probability that an allele frequency equals f for the hypothesized allele, n is the total read depth for an SNP marker, k is the number of reads supporting an alternate allele and (n−k) is the number of reads supporting a reference allele. The function Beta represents the probability model fit to the observed read count data for the hypothesized alleles. For example, the probability model may be a Gaussian probability density function or a beta-binomial distribution.

In step 204, the probability for each possible alternate allele dosage is calculated. For example, a tetraploid organism has five possibilities corresponding to allele dosages of 0, 1, 2, 3, and 4. For this example, these may be calculated as follows.

For the probability, p₀, of allele dosage 0:

$${p}_{0} = {\int }_{f = 0}^{0.125} {f}_{A P P} ( {p}_{H} = f ) \qquad ( 2 )$$

For the probability, p₁, of allele dosage 1:

$${p}_{1} = {\int }_{f = 0.125}^{0.375} {f}_{A P P} ( {p}_{H} = f ) \qquad ( 3 )$$

For the probability, p₂, of allele dosage 2:

$${p}_{2} = {\int }_{f = 0.375}^{0.625} {f}_{A P P} ( {p}_{H} = f ) \qquad ( 4 )$$

For the probability, p₃, of allele dosage 3:

$${p}_{3} = {\int }_{f = 0.625}^{0.875} {f}_{A P P} ( {p}_{H} = f ) \qquad ( 5 )$$

For the probability, p₄, of allele dosage 4:

$${p}_{4} = {\int }_{f = 0.875}^{1} {f}_{A P P} ( {p}_{H} = f ) \qquad ( 6 )$$

The limits of integration in equations (2) through (6) are default values for a ploidy of 4. Ploidy allele frequency boundary parameters may be used to set the limits of integration. The ploidy allele frequency boundary parameter is the allele frequency value of a boundary in the a posteriori probability distribution f_{APP} (p_{H}=f) for genotyping a variant associated with a specific marker. The number of ploidy allele frequency boundary parameters is the ploidy value plus 2. Examples of default values of the ploidy allele frequency boundary parameters for different ploidies are given in Table 1.

TABLE 1

| Ploidy | Ploidy Allele Frequency Boundary Parameters (Default Values) |
|---|---|
| 4 | 0, 0.125, 0.375, 0.625, 0.875, 1.0 |
| 6 | 0, 0.08333333, 0.25, 0.41666667, 0.58333333, 0.75, |
| | 0.91666667, 1.0 |
| 8 | 0, 0.0625, 0.1875, 0.3125, 0.4375, 0.5625, 0.6875, 0.8125, |
| | 0.9375, 1.0 |

The ploidy allele frequency boundary parameters, and the related limits of integration in equations (2) through (6), may be set to other values. For example, the limits of integration may be customized for each SNP marker. For example in some organisms, the ploidy of a given marker may be different than the ploidy for the organism as a whole. For instance, the ploidy of a given marker may be 4, while the ploidy of the organism is 6. The ploidy allele frequency boundaries for the given marker may be set to the values corresponding to a ploidy of 4, while the ploidy allele frequency boundaries for the other markers in the organism may be set to the values corresponding to a ploidy of 4. In another example, when the ploidy allele frequency boundary for a particular marker intersects a cluster of observed allele frequency values associated with the particular marker, the value of the ploidy allele frequency boundary parameter for the marker may be adjusted so that allele frequency values associated with the marker are on the same side of the boundary. A user may set the values of the ploidy allele frequency boundary parameters.

In step 206, the allele dosage i with the highest probability value is selected.

$$i = {Max}_{i = \{ 0 , 1 , 2 , 3 , 4 \}} \{ {p}_{i} \} \qquad ( 7 )$$

The estimated allele dosage is the one which has the highest probability value. The estimated allele dosage may indicate the genotype associated with the marker and sample.

In step 208, the allele dosage quality may be calculated. The estimated allele dosage quality is the probability of estimating an incorrect allele dosage. The probability of incorrect allele dosage may be calculated as −10 log₁₀ (summation of probabilities supporting other allele dosages). For example, if the estimated allele dosage is 1, then the allele dosage quality is given by:

$$Allele dosage quality = - 10 {log}_{10} ( {p}_{0} + {p}_{2} + {p}_{3} + {p}_{4} )$$

A threshold may be applied to the allele dosage quality to determine whether a genotype call should be made. A minimum variant score parameter may provide a threshold value for allele dosage quality. If the allele dosage quality score is lower than that the minimum variant score, then the genotype call will be a “NO CALL”. The minimum variant score may be set to a value greater than 0. For example, the minimum variant score may be set to a default value of 10. The minimum variant score parameter may be set by the user.

A threshold may be applied to the coverage for the location of the allele of the SNP marker to determine whether a genotype call should be made. A minimum coverage parameter may provide a threshold value for the coverage. The minimum coverage parameter is greater than zero. For example, the minimum coverage parameter may have a default value of 10*ploidy. For example, for a tetraploid (ploidy=4), the minimum coverage parameter may be set to 40. If the coverage is lower than minimum coverage parameter value, then a “NO CALL” is assigned. The minimum coverage parameter may be set to an integer value greater than 0 by the user. The minimum coverage parameter may be set to a custom value for a particular marker. There may be different minimum coverage parameters for different markers.

Comparative studies for detection of allele dosage using the present method and other methods were performed. Table 2 shows the organisms, number of samples multiplexed and number of markers targeted by the AgriSeq HTS Library Kit panels.

TABLE 2

| Organism | Ploidy | Samples | Markers |
|---|---|---|---|
| Potato | 4 | 96 | 417 |
| Chrysanthemum | 6 | 96 | 1023 |

The results in Table 3 show comparisons of allele dosage calls using the present method (“This Method”) applied to read count data for SNPs detected by variant calling, as described with respect to FIGS. 1 and 2, and other methods to provide benchmarking data. Column A of Table 3 shows the comparison results with the Fitpoly method applied to frequency data obtained from microarray experiments. (For Fitpoly method, see e.g., www.bmcbioinformatics.biomedcentral.com/articles/10.1186/s12859-019-2703-y). Column B of Table 3 shows the comparison results with the Fitpoly method applied to read count data for SNPs detected by variant calling, such as the read count data generated by applying steps 102, 104 and 106 of FIG. 1. The numbers in parentheses are the numbers of allele dosage calls using the respective methods. The “NO CALL” instances were removed from the results prior to the comparison calculations. The percent value gives the ratio of allele dosage calls produced by the respective methods, expressed as a percentage.

TABLE 3

| | A. This Method vs Fitpoly | B. This Method vs |
|---|---|---|
| | (Microarray Frequency | Fitpoly |
| Organism | Data) | (Read Count Data) |
| Potato | 97.3% (21908/22512) | 99.2% (34689/34972) |
| Chrysanthemum | 92.4% (67057/72385) | 98.1% (85045/91158) |

The results of TABLE 3 show that present method is generally agrees with the benchmark data generated using the Fitpoly method applied to microarray frequency data and the Fitpoly method applied to read count data. The Fitpoly method is based on clustering data from multiple samples and requires at least 10*(ploidy+1) samples for each marker, as used in the above comparisons in columns A and B. However, the Fitpoly method cannot determine allele dosage on a per sample basis and is ineffective for lower numbers of samples or for monomorphic markers in the samples. The present method can determine allele dosages on a per sample basis, so it is effective when there is a low number of samples or when a particular marker is monomorphic in all the multiplexed samples. TABLE 4 shows the average call rate by Fitpoly when lower number of samples were used. For Potato and Chrysanthemum, the average call rate by Fitpoly is zero until the data for at least 50 (10*(4+1)) and 70 (10*(6+1)) samples are provided, respectively. In contrast, the present method shows high call rates, well above 90%, for the number of samples as low as 12.

TABLE 4

| Number of | Fitpoly | Fitpoly | This Method | This Method |
|---|---|---|---|---|
| Samples | Potato | Chrysanthemum | Potato | Chrysanthemum |
| 12 | 0 | 0 | 99.28% | 97.80% |
| 24 | 0 | 0 | 98.87% | 93.75% |
| 36 | 0 | 0 | 97.99% | 95.07% |
| 48 | 0 | 0 | 97.83% | 93.75% |
| 60 | 95.90% | 0 | 98.47% | 91.39% |
| 72 | 97.20% | 5.77% | 98.28% | 93.69% |

Table 5 shows results of a comparison of the Updog method with benchmark data obtained using the Fitpoly method. (See e.g., Gerard, D. et al. Genotyping Polyploids from Messy Sequencing Data, Genetics. 2018 November; 210(3): 789-807. doi: 10.1534/genetics.118.301468. Epub 2018 September 5. PMID: 30185430; PMCID: PMC6218231). For this comparison, the Fitpoly method applied to microarray frequency data was compared to the Updog method applied to applied to read count data for SNPs detected by variant calling, such as the read count data generated by applying steps 102, 104 and 106 of FIG. 1. The results in Table 5 show comparisons of allele dosage calls using the Fitpoly method applied to microarray frequency data to the Updog method applied to applied to read count data. The numbers in parentheses are the numbers of allele dosage calls using the respective methods. The “NO CALL” instances were removed from the results prior to the comparison calculations. The percent value gives the ratio of allele dosage calls produced by the respective methods, expressed as a percentage.

TABLE 5

| | Fitpoly (Microarray Frequency Data) |
|---|---|
| Organism | vs Updog (Read Count Data) |
| Potato | 44.77% (9999/22306) |
| Chrysanthemum | 45.05% (32819/71189) |

The results of Table 5 show that the Updog method, which was primarily designed for messy sequencing data, performs poorly when compared with benchmark data obtained by applying the Fitpoly method to microarray frequency data, since the agreement of allele dosage calls with the benchmark data is less than 50%. In contrast, agreement of allele dosage calls of the present method with the benchmark data is over 90%, as shown in Table 3. Thus, the present method produced improved results over the Updog method, as demonstrated by comparison of Table 3 and Table 5.

The embodiments disclosed herein may achieve more accurate genotyping calls for polyploid organisms relative to conventional approaches. The greater accuracy relative to the conventional approach Fitpoly is demonstrated by the results described with respect to Table 3. The greater accuracy relative to the conventional approach Updog is demonstrated by the results described with respect to Table 4. These conventional approaches suffer from a number of technical problems and limitations, including requiring multiple samples to support the determination of allele dosages. The conventional approach cannot determine allele dosage on a per sample basis, and is ineffective for lower numbers of samples or for monomorphic markers in the samples. The embodiments disclosed herein can determine allele dosages on a per sample basis and are effective when there is a low number of samples or when a particular marker is monomorphic in all the multiplexed samples.

Various ones of the embodiments disclosed herein may improve upon conventional approaches to achieve the technical advantages of improved accuracy of detection of allele dosages and genotyping of polyploid organisms. Technical advantages of the embodiments described herein include providing allele dosages for markers in an individual sample, without considering information from other samples in a multiplexed run. The embodiments described herein are not susceptible to bias in allele dosage estimation when there is a low number of samples or if a particular marker is monomorphic in all the multiplexed samples. Technical advantages of the embodiments described herein include allowing for marker level parameter customization, which enables greater accuracy in the estimate of allele dosage per marker. Such technical advantages are not achievable by routine and conventional approaches, and users of systems and methods including such embodiments may benefit from these advantages. The technical features of the embodiments disclosed herein are thus unconventional in the field of deriving a genotype of a polyploid organism.

Accordingly, the embodiments of the present disclosure serve a technical purpose, such as deriving a genotype estimate on a per sample basis for a polyploid organism. In particular, the present disclosure provides technical solutions to technical problems, including but not limited to improving the accuracy of allele dosage estimates and genotyping for polyploid organisms.

In various embodiments, nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, fluorescent-based detection systems, single molecule methods, etc.

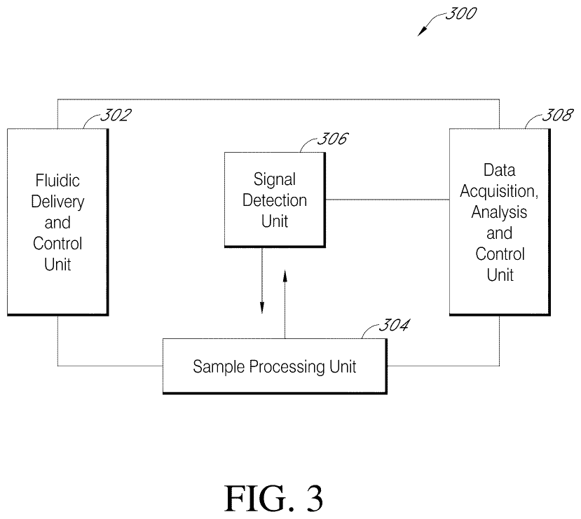

Various embodiments of nucleic acid sequencing platforms, such as a nucleic acid sequencer, can include components as displayed in the block diagram of FIG. 3. According to various embodiments, sequencing instrument 300 can include a fluidic delivery and control unit 302, a sample processing unit 304, a signal detection unit 306, and a data acquisition, analysis and control unit 308. Various embodiments of instrumentation, reagents, libraries and methods used for next generation sequencing are described in U.S. Patent Application Publication No. 2009/0127589 and No. 2009/0026082. Various embodiments of instrument 300 can provide for automated sequencing that can be used to gather sequence information from a plurality of sequences in parallel, such as substantially simultaneously.

In various embodiments, the fluidics delivery and control unit 302 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 304 can include a sample chamber, such as flow cell, a substrate, a microarray, a multi-well tray, or the like. The sample processing unit 304 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 306 can include an imaging or detection sensor. For example, the imaging or detection sensor can include a CCD, a CMOS, an ion sensor, such as an ion sensitive layer overlying a CMOS, a current detector, or the like. The signal detection unit 306 can include an excitation system to cause a probe, such as a fluorescent dye, to emit a signal. The excitation system can include an illumination source, such as arc lamp, a laser, a light emitting diode (LED), or the like. In particular embodiments, the signal detection unit 306 can include optics for the transmission of light from an illumination source to the sample or from the sample to the imaging or detection sensor. Alternatively, the signal detection unit 306 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 308 can monitor various system parameters. The system parameters can include temperature of various portions of instrument 300, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 300 can be used to practice variety of sequencing methods including ligation-based methods, sequencing by synthesis, single molecule methods, nanopore sequencing, and other sequencing techniques.

In various embodiments, the sequencing instrument 300 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 300 can obtain the sequence information from a single nucleic acid molecule or from a group of substantially identical nucleic acid molecules.

In various embodiments, sequencing instrument 300 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

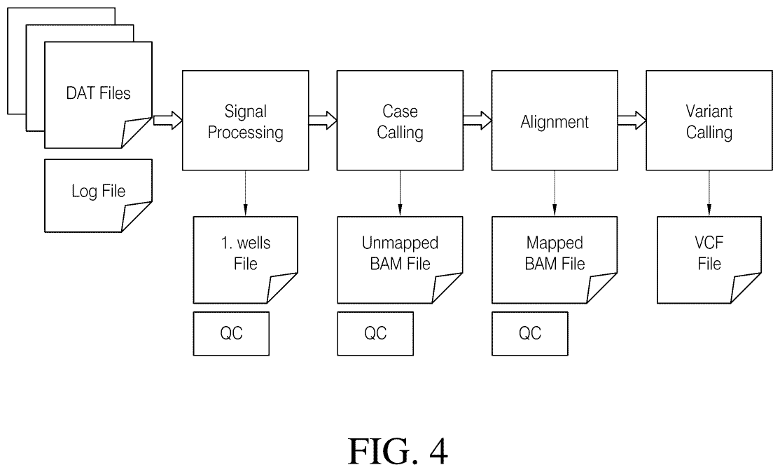

FIG. 4 is a block diagram of an analysis pipeline for signal data obtained from a nucleic acid sequencing instrument. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases, and average read length as quality control (QC) measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

The variant calling step may include detecting single-nucleotide polymorphisms (SNPs), insertions and deletions (InDels), multi-nucleotide polymorphisms (MNPs), and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which is incorporated by reference herein in its entirety.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Hash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Python, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

### EXAMPLES

Example 1 is a method for determining a genotype of a sample of a polyploid organism, including: amplifying nucleic acid sequences at targeted locations in a sample genome by a panel targeting a plurality of SNP markers of the sample to generate a plurality of sequence reads; mapping the plurality of sequence reads to a reference genome for the polyploid organism to produce a plurality of aligned sequence reads; detecting variants in the aligned sequence reads to produce a plurality of detected variants, wherein the detected variants include detected SNP's corresponding to the SNP markers of the sample; determining a probability for each alternate allele dosage of a plurality of possible allele dosages for a corresponding detected SNP, wherein a number of the possible allele dosages is equal to a ploidy of the SNP marker plus one; and selecting the alternate allele dosage with a maximum probability value to provide an estimated allele dosage corresponding to the SNP marker of the sample, wherein the estimated allele dosage is indicative of the genotype for the SNP marker of the sample.

Example 2 includes the subject matter of Example 1, and further includes determining an allele dosage quality based on a summation of probabilities supporting other possible allele dosages.

Example 3 includes the subject matter of Example 2, and further specifies that the determining the allele dosage quality further comprises calculating a log₁₀ of the summation of probabilities and multiplying the log₁₀ of the summation of probabilities by (−10).

Example 4 includes the subject matter of Example 2, and further includes applying a threshold to the allele dosage quality to determine whether a genotype call will be made, wherein if the allele dosage quality is less than the threshold then the genotype call will be a “NO CALL”.

Example 5 includes the subject matter of Example 1, and further includes applying a threshold to a coverage for a location of the SNP marker to determine whether a genotype call will be made, wherein if the coverage is less than the threshold then the genotype call will be a “NO CALL”.

Example 6 includes the subject matter of Example 1, and further specifies that the determining the probability for each alternate allele dosage is based on an a posteriori probability distribution of allele frequencies for a hypothesized allele.

Example 7 includes the subject matter of Example 6, and further specifies that the determining the probability for each alternate allele dosage further comprises integrating the a posteriori probability distribution of allele frequencies between limits of integration, wherein allele frequency boundary parameters set the limits of integration for each possible allele dosage corresponding to the SNP marker.

Example 8 includes the subject matter of Example 7, and further specifies that a number of the allele frequency boundary parameters is equal to the ploidy of the SNP marker plus two.

Example 9 includes the subject matter of Example 7, and further specifies that the allele frequency boundary parameters when the ploidy of the SNP marker is 4 have values of 0, 0.125, 0.375, 0.625, 0.875, and 1.0.

Example 10 includes the subject matter of Example 7, and further specifies that the allele frequency boundary parameters when the ploidy of the SNP marker is 6 have values of 0, 0.08333333, 0.25, 0.41666667, 0.58333333, 0.75, 0.91666667, and 1.0.

Example 11 includes the subject matter of Example 7, and further specifies that the allele frequency boundary parameters when the ploidy of the SNP marker is 8 have values of 0, 0.0625, 0.1875, 0.3125, 0.4375, 0.5625, 0.6875, 0.8125, 0.9375, and 1.0.

Example 12 includes the subject matter of Example 7, and further specifies that values of the allele frequency boundary parameters are adjustable on a per marker basis.

Example 13 includes the subject matter of Example 5, and further specifies that the threshold is adjustable on a per marker basis.

Example 14 includes the subject matter of Example 1, and further includes determining the estimated allele dosage for each SNP marker of each sample of a plurality of samples from one or more polyploid organisms, wherein the plurality of sequence reads are produced for the plurality of samples by a single sequencing run.

Example 15 is a system for determining a genotype of a sample of a polyploid organism, including: a machine-readable memory; and a processor configured to execute machine-readable instructions, which are configured to, when executed by the processor, cause the system to perform steps, comprising: receiving, at the processor, a plurality of sequence reads produced by amplifying nucleic acid sequences at targeted locations in a sample genome by a panel targeting a plurality of SNP markers of the sample; mapping the plurality of sequence reads to a reference genome for the polyploid organism to produce a plurality of aligned sequence reads; detecting variants in the aligned sequence reads to produce a plurality of detected variants, wherein the detected variants include detected SNP's corresponding to the SNP markers of the sample; determining a probability for each alternate allele dosage of a plurality of possible allele dosages for a corresponding detected SNP, wherein a number of the possible allele dosages is equal to a ploidy of the SNP marker plus one; and selecting the alternate allele dosage with a maximum probability value to provide an estimated allele dosage corresponding to the SNP marker of the sample, wherein the estimated allele dosage is indicative of the genotype for the SNP marker of the sample.

Example 16 includes the subject matter of Example 15, and further specifies that the steps further include determining an allele dosage quality based on a summation of probabilities supporting other possible allele dosages.

Example 17 includes the subject matter of Example 16, and further specifies that the determining the allele dosage quality further comprises calculating a log₁₀ of the summation of probabilities and multiplying the log₁₀ of the summation of probabilities by (−10).

Example 18 includes the subject matter of Example 16, and further specifies that the steps further include applying a threshold to the allele dosage quality to determine whether a genotype call will be made, wherein if the allele dosage quality is less than the threshold then the genotype call will be a “NO CALL”.

Example 19 includes the subject matter of Example 15, and further specifies that the steps further include applying a threshold to a coverage for a location of the SNP marker to determine whether a genotype call will be made, wherein if the coverage is less than the threshold then the genotype call will be a “NO CALL”.

Example 20 includes the subject matter of Example 15, and further specifies that the determining the probability for each alternate allele dosage is based on an a posteriori probability distribution of allele frequencies for a hypothesized allele.

Example 21 includes the subject matter of Example 20, and further specifies that the determining the probability for each alternate allele dosage further comprises integrating the a posteriori probability distribution of allele frequencies between limits of integration, wherein allele frequency boundary parameters set the limits of integration for each possible allele dosage corresponding to the SNP marker.

Example 22 includes the subject matter of Example 21, and further specifies that a number of the allele frequency boundary parameters is equal to the ploidy of the SNP marker plus two.

Example 23 includes the subject matter of Example 21, and further specifies that the allele frequency boundary parameters when the ploidy of the SNP marker is 4 have values of 0, 0.125, 0.375, 0.625, 0.875, and 1.0.

Example 24 includes the subject matter of Example 21, and further specifies that the allele frequency boundary parameters when the ploidy of the SNP marker is 6 have values of 0, 0.08333333, 0.25, 0.41666667, 0.58333333, 0.75, 0.91666667, and 1.0.

Example 25 includes the subject matter of Example 21, and further specifies that the allele frequency boundary parameters when the ploidy of the SNP marker is 8 have values of 0, 0.0625, 0.1875, 0.3125, 0.4375, 0.5625, 0.6875, 0.8125, 0.9375, and 1.0.

Example 26 includes the subject matter of Example 21, and further specifies that values of the allele frequency boundary parameters are adjustable on a per marker basis.

Example 27 includes the subject matter of Example 19, and further specifies that the threshold is adjustable on a per marker basis.

Example 28 includes the subject matter of Example 15, and further specifies that the steps further include determining the estimated allele dosage for each SNP marker of each sample of a plurality of samples from one or more polyploid organisms, wherein the plurality of sequence reads are produced for the plurality of samples by a single sequencing run.

Example 29 is a non-transitory machine-readable storage medium comprising instructions which are configured to, when executed by a processor, cause the processor to perform a method for estimating quality values of nucleotide base calls, including: receiving, at the processor, a plurality of sequence reads produced by amplifying nucleic acid sequences at targeted locations in a sample genome by a panel targeting a plurality of SNP markers of the sample; mapping the plurality of sequence reads to a reference genome for the polyploid organism to produce a plurality of aligned sequence reads; detecting variants in the aligned sequence reads to produce a plurality of detected variants, wherein the detected variants include detected SNP's corresponding to the SNP markers of the sample; determining a probability for each alternate allele dosage of a plurality of possible allele dosages for a corresponding detected SNP, wherein a number of the possible allele dosages is equal to a ploidy of the SNP marker plus one; and selecting the alternate allele dosage with a maximum probability value to provide an estimated allele dosage corresponding to the SNP marker of the sample, wherein the estimated allele dosage is indicative of the genotype for the SNP marker of the sample.

Example 30 includes the subject matter of Example 29, further including instructions which cause the processor to perform the method, and further includes determining an allele dosage quality based on a summation of probabilities supporting other possible allele dosages.

Example 31 includes the subject matter of Example 30, and further specifies that the determining the allele dosage quality further comprises calculating a log₁₀ of the summation of probabilities and multiplying the log₁₀ of the summation of probabilities by (−10).

Example 32 includes the subject matter of Example 30, further including instructions which cause the processor to perform the method, and further includes applying a threshold to the allele dosage quality to determine whether a genotype call will be made, wherein if the allele dosage quality is less than the threshold then the genotype call will be a “NO CALL”.

Example 33 includes the subject matter of Example 29, further including instructions which cause the processor to perform the method, and further includes applying a threshold to a coverage for a location of the SNP marker to determine whether a genotype call will be made, wherein if the coverage is less than the threshold then the genotype call will be a “NO CALL”.

Example 34 includes the subject matter of Example 29, and further specifies that the determining the probability for each alternate allele dosage is based on an a posteriori probability distribution of allele frequencies for a hypothesized allele.

Example 35 includes the subject matter of Example 34, and further specifies that the determining the probability for each alternate allele dosage further comprises integrating the a posteriori probability distribution of allele frequencies between limits of integration, wherein allele frequency boundary parameters set the limits of integration for each possible allele dosage corresponding to the SNP marker.

Example 36 includes the subject matter of Example 35, and further specifies that a number of the allele frequency boundary parameters is equal to the ploidy of the SNP marker plus two.

Example 37 includes the subject matter of Example 35, and further specifies that the allele frequency boundary parameters when the ploidy of the SNP marker is 4 have values of 0, 0.125, 0.375, 0.625, 0.875, and 1.0.

Example 38 includes the subject matter of Example 35, and further specifies that the allele frequency boundary parameters when the ploidy of the SNP marker is 6 have values of 0, 0.08333333, 0.25, 0.41666667, 0.58333333, 0.75, 0.91666667, and 1.0.

Example 39 includes the subject matter of Example 35, and further specifies that the allele frequency boundary parameters when the ploidy of the SNP marker is 8 have values of 0, 0.0625, 0.1875, 0.3125, 0.4375, 0.5625, 0.6875, 0.8125, 0.9375, and 1.0.

Example 40 includes the subject matter of Example 35, and further specifies that values of the allele frequency boundary parameters are adjustable on a per marker basis.

Example 41 includes the subject matter of Example 33, and further specifies that the threshold is adjustable on a per marker basis.

Example 42 includes the subject matter of Example 29, further including instructions which cause the processor to perform the method, and further includes determining the estimated allele dosage for each SNP marker of each sample of a plurality of samples from one or more polyploid organisms, wherein the plurality of sequence reads are produced for the plurality of samples by a single sequencing run.

What is claimed is:

1. A method for determining a genotype of a sample of a polyploid organism, comprising:
amplifying nucleic acid sequences at targeted locations in a sample genome by a panel targeting a plurality of SNP markers of the sample to generate a plurality of sequence reads;
mapping the plurality of sequence reads to a reference genome for the polyploid organism to produce a plurality of aligned sequence reads;
detecting variants in the aligned sequence reads to produce a plurality of detected variants, wherein the detected variants include detected SNP's corresponding to the SNP markers of the sample;
determining a probability for each alternate allele dosage of a plurality of possible allele dosages for a corresponding detected SNP, wherein a number of the possible allele dosages is equal to a ploidy of the SNP marker plus one; and
selecting the alternate allele dosage with a maximum probability value to provide an estimated allele dosage corresponding to the SNP marker of the sample, wherein the estimated allele dosage is indicative of the genotype for the SNP marker of the sample.

2. The method of claim 1, further comprising determining an allele dosage quality based on a summation of probabilities supporting other possible allele dosages.

3. The method of claim 2, wherein the determining the allele dosage quality further comprises calculating a log₁₀ of the summation of the probabilities and multiplying the log₁₀ of the summation of probabilities by (−10).

4. The method of claim 2, further comprising applying a threshold to the allele dosage quality to determine whether a genotype call will be made, wherein if the allele dosage quality is less than the threshold then the genotype call will be a “NO CALL”.

5. The method of claim 1, further comprising applying a threshold to a coverage for a location of the SNP marker to determine whether a genotype call will be made, wherein if the coverage is less than the threshold then the genotype call will be a “NO CALL”.

6. The method of claim 1, wherein the determining the probability for each alternate allele dosage is based on an a posteriori probability distribution of allele frequencies for a hypothesized allele.

7. The method of claim 6, wherein the determining the probability for each alternate allele dosage further comprises integrating the a posteriori probability distribution of allele frequencies between limits of integration, wherein allele frequency boundary parameters set the limits of integration for each possible allele dosage corresponding to the SNP marker.

8. The method of claim 7, wherein values of the allele frequency boundary parameters are adjustable on a per marker basis.

9. The method of claim 5, wherein the threshold is adjustable on a per marker basis.

10. The method of claim 1, further comprising determining the estimated allele dosage for each SNP marker of each sample of a plurality of samples from one or more polyploid organisms, wherein the plurality of sequence reads are produced for the plurality of samples by a single sequencing run.

11. A system for determining a genotype of a sample of a polyploid organism, comprising:
a machine-readable memory; and
a processor configured to execute machine-readable instructions, which are configured to, when executed by the processor, cause the system to perform steps, comprising:
receiving, at the processor, a plurality of sequence reads produced by amplifying nucleic acid sequences at targeted locations in a sample genome by a panel targeting a plurality of SNP markers of the sample;
mapping the plurality of sequence reads to a reference genome for the polyploid organism to produce a plurality of aligned sequence reads;
detecting variants in the aligned sequence reads to produce a plurality of detected variants, wherein the detected variants include detected SNP's corresponding to the SNP markers of the sample;
determining a probability for each alternate allele dosage of a plurality of possible allele dosages for a corresponding detected SNP, wherein a number of the possible allele dosages is equal to a ploidy of the SNP marker plus one; and
selecting the alternate allele dosage with a maximum probability value to provide an estimated allele dosage corresponding to the SNP marker of the sample, wherein the estimated allele dosage is indicative of the genotype for the SNP marker of the sample.

12. The system of claim 11, wherein the steps further include determining an allele dosage quality based on a summation of probabilities supporting other possible allele dosages.

13. The system of claim 12, wherein the determining the allele dosage quality further comprises calculating a log₁₀ of the summation of probabilities and multiplying the log₁₀ of the summation of probabilities by (−10).

14. The system of claim 12, wherein the steps further include applying a threshold to the allele dosage quality to determine whether a genotype call will be made, wherein if the allele dosage quality is less than the threshold then the genotype call will be a “NO CALL”.

15. The system of claim 11, wherein the steps further include applying a threshold to a coverage for a location of the SNP marker to determine whether a genotype call will be made, wherein if the coverage is less than the threshold then the genotype call will be a “NO CALL”.

16. The system of claim 11, wherein the determining the probability for each alternate allele dosage is based on an a posteriori probability distribution of allele frequencies for a hypothesized allele.

17. The system of claim 16, wherein the determining the probability for each alternate allele dosage further comprises integrating the a posteriori probability distribution of allele frequencies between limits of integration, wherein allele frequency boundary parameters set the limits of integration for each possible allele dosage corresponding to the SNP marker.

18. The system of claim 17, wherein values of the allele frequency boundary parameters are adjustable on a per marker basis.

19. The system of claim 15, wherein the threshold is adjustable on a per marker basis.

20. The system of claim 11, wherein the steps further include determining the estimated allele dosage for each SNP marker of each sample of a plurality of samples from one or more polyploid organisms, wherein the plurality of sequence reads are produced for the plurality of samples by a single sequencing run.
